(19) United States
(12) Patent Application Publication
(10) Pub. No.: US 2024/0024037 A1
(43) Pub. Date: Jan. 25, 2024

(54) SYSTEMS AND METHODS OF GENERATING RECONSTRUCTED IMAGES FOR INTERVENTIONAL MEDICAL PROCEDURES

(71) Applicant: KONINKLIJKE PHILIPS N.V., EINDHOVEN (NL)

(72) Inventors: Towa Matsumura, Clinton, MA (US); Eduardo Ortiz Vazquez, Cambridge, MA (US); Maarten Heye, Best (NL); Andrea Laghi, Melrose, MA (US)

(21) Appl. No.: 18/265,470

(22) PCT Filed: Dec. 6, 2021
(86) PCT No.: PCT/EP2021/084408

§ 371 Date: Jun. 6, 2023

Related U.S. Application Data
(60) Provisional application No. 63/122,756, filed on Dec. 8, 2020.

(51) Int. Cl. A61B 34/20; A61B 8/08; A61B 8/00; G06T 15/08; G06V 20/50; G06V 10/44; G06V 10/98

(52) U.S. Cl. CPC A61B 34/20; A61B 8/0841; A61B 8/463; A61B 8/467; A61B 8/483; A61B 8/0883; A61B 8/54; G06T 15/08; G06V 20/50; G06V 10/44; G06V 10/98; A61B 2034/2063; A61B 2034/2065; G06T 2200/24; G06V 2201/031

(57) ABSTRACT

Systems and methods for providing image guidance during interventional medical procedures are disclosed. Methods involve receiving a user selection of an interventional medical procedure at an adaptive user interface, acquiring volumetric image data of a region of interest using 3D ultrasound, and identifying at least one anatomical landmark within the volumetric image data specific to the selected procedure. Methods further involve reconstructing one or more ultrasound images along predefined image planes necessary for performing the selected procedure. The reconstructed images are displayed on the user interface for providing real-time guidance to a clinician performing the procedure, which may involve deploying one or more interventional instruments within the region of interest.

### TECHNICAL FIELD

This application relates to systems configured to generate ultrasound images necessary for performing various interventional medical procedures. More specifically, this application relates to systems and methods for generating reconstructed views of an anatomical feature with improved efficiency and accuracy during an interventional medical procedure while reducing reliance on manual input.

### BACKGROUND

Extremely accurate positioning of an interventional device, such as a catheter, is essential for many medical procedures, e.g., interventional cardiology procedures. For this reason, the clinicians performing such procedures often rely on high-resolution ultrasound imaging for precise, real-time guidance. Ultrasound imaging is indeed helpful for guiding the placement of interventional devices, such as stents, but in order to position such devices with high precision, the acquisition of multiple different views of at least one anatomical feature, e.g., heart valve, is typically needed over the course of a given procedure. Existing ultrasound technologies can be used to obtain images along the planes necessary to guide clinicians through the image acquisition process, but such technologies require substantial and often difficult manual manipulation of an ultrasound probe and system. Clinicians must therefore demonstrate a consistent mastery of ultrasound transducer tip placement and manipulation of 2D planes from a 3D volume to obtain the best-possible views of an anatomical feature. Such skills usually require repetitive adjustments on behalf of the clinician throughout a given operation, which further increase the difficulty of performing interventional procedures within a small anatomical volume that may significantly increase procedure time or be resistant to effective treatment. New ultrasound systems configured to improve the accuracy and consistency of interventional procedures in a computationally efficient manner are needed.

### SUMMARY

Systems and methods for providing more efficient and accurate ultrasound image guidance during interventional medical procedures are disclosed. Examples may involve automating the ultrasound data acquisition steps necessary to obtain anatomical images along particular image planes in real time. Such examples may involve using 3D ultrasound to acquire volumetric image data of a region of interest, transmitting the acquired volumetric image data to a processor configured to identify anatomical landmarks within the volumetric data, and automatically reconstructing specific views of one or more regions of interest based on the identified anatomical landmarks. The reconstructed views can then be used to guide a clinician performing an interventional medical procedure, which may involve deploying one or more interventional instruments, such as catheters and/or implants, within a specific anatomical location, such as a blood vessel and/or valve. Unlike preexisting systems, a user interface coupled with the ultrasound acquisition system can be configured to display procedure-specific graphics, user-input options and/or procedural instructions in response to receiving a procedure selection and/or in response to the system detecting one or more anatomical features. The display shown on the user interface may therefore be tailored to a currently selected procedure. The examples described herein may be implemented independently of the position and orientation of an ultrasound transducer relative to a region of interest, provided the region of interest is adequately captured in the initially-acquired volumetric image data.

In accordance with at least one example disclosed herein, an ultrasound system may include a user interface configured to receive a user input indicating a selection of an interventional medical procedure. The system may also include an ultrasound probe configured to transmit ultrasound signals at a target region and receive echoes responsive to the ultrasound signals and generate radio frequency (RF) data corresponding to the echoes. The system may also include an image processor configured to generate image data from the RF data. Non-limiting examples of such image data can include per-channel data, pre-beamformed data, post-beamformed data, log-detected data, scan converted data, and processed echo data in 2D and/or 3D. The system can also include an anatomical recognition processor configured to receive the image data and identify an anatomical landmark within the image data. The system can also include an image reconstruction processor configured to generate a planar ultrasound image along an image plane relevant to the selected interventional medical procedure based on the anatomical landmark identified within the image data. The user interface can be configured to display the planar ultrasound image during the interventional medical procedure.

In some examples, the interventional medical procedure includes a cardiovascular valve clip procedure, an annuloplasty procedure or a left atrial appendage occlusion procedure. In some embodiments, the image data includes 3D data acquired via a volumetric imaging mode. In some examples, the user interface is further configured to receive an indication of procedure-specific implant locations. In some embodiments, the image reconstruction processor is configured to generate at least two planar ultrasound images along at least two image planes relevant to the selected interventional medical procedure based on the anatomical landmark identified within the image data and the interventional devices required to perform the interventional medical procedure. In some examples, the user interface is configured to display the images sequentially as the interventional medical procedure is being performed. In some embodiments, the system further includes a controller configured to cause the ultrasound probe to cease transmitting ultrasound signals except for the ultrasound signals required to generate the planar ultrasound image plane relevant to the selected interventional medical procedure.

In some examples, the user interface is configured to receive a confirmation that a step in the selected interventional medical procedure has been performed. In some embodiments, the user interface is configured to display a second planar ultrasound image based on the confirmation, the second planar ultrasound image being necessary to perform a subsequent step in the selected interventional medical procedure. In some examples, the image reconstruction processor is configured to generate the planar ultrasound image along the image plane relevant to the selected interventional medical procedure in real time. In some embodiments, the image reconstruction processor is configured to optimize a scan sequence for the image plane relevant to the selected interventional medical in an interval or periodic basis. In some examples, the anatomical recognition processor is further configured to generate an indication that the image data is insufficient to identify the anatomical landmark. In some embodiments, the image plane is a 2D image plane.

In accordance with at least one example disclosed herein, a method can involve receiving a user input indicating a selection of an interventional medical procedure and obtaining image data by transmitting ultrasound signals at a target region and receiving echoes responsive to the ultrasound signals from the target region. The method may involve automatically identifying an anatomical landmark within the image data. The method may further involve automatically generating a planar ultrasound image along an image plane relevant to the selected interventional medical procedure based on the anatomical landmark identified within the image data and displaying the planar ultrasound image during the interventional medical procedure.

In some examples, the interventional medical procedure comprises a cardiovascular valve clip procedure, an annuloplasty procedure, or a left atrial appendage occlusion procedure. In some examples, the method may further involve receiving an indication of procedure-specific implant locations. In some examples, the method further involves ceasing obtaining image data except for the image data required to generate the planar ultrasound image along the image plane relevant to the selected interventional medical procedure. In some examples, the method further involves receiving a confirmation that a step in the selected interventional medical procedure has been performed. In some examples, the method further involves displaying a second planar ultrasound image based on the confirmation, the second planar ultrasound image being necessary to perform a subsequent step in the selected interventional medical procedure. Examples may involve generating the planar ultrasound image along the image plane relevant to the selected interventional medical procedure in real time. Examples may involve optimizing a scan sequence for the image plane relevant to the selected interventional medical procedure in an interval or periodic basis. In some examples, the method may involve generating an indication that the image data is insufficient to identify the anatomical landmark.

### BRIEF DESCRIPTION OF THE DRAWINGS

FIG. 1 is a block diagram of an ultrasound imaging system arranged according to principles of the present disclosure.

FIG. 2 is a block diagram illustrating an example processor according to principles of the present disclosure.

FIG. 3 is a graphical depiction of a scan sequence optimization scheme implemented according to examples of the present disclosure.

FIG. 4 is a flow chart of an example process implemented according to embodiments of the present disclosure.

FIG. 5 is a flow chart of an example method implemented according to principles of the present disclosure.

FIG. 6 illustrates an example image view necessary for performing a cardiovascular valve clip procedure.

FIG. 7 illustrates an example image view necessary for performing the cardiovascular valve clip procedure of FIG. 6.

FIG. 8 is a graphic displayed on a user interface pursuant to performing the cardiovascular valve clip procedure of FIG. 6.

FIG. 9 is a flow chart of a method implemented for valve clip image guidance in accordance with principles of the present disclosure.

FIG. 10 illustrates example image views necessary for performing a cardiovascular annuloplasty procedure.

FIG. 11 is a graphic displayed on a user interface pursuant to performing the annuloplasty procedure of FIG. 10 in accordance with embodiments of the present disclosure.

FIG. 12 is a volumetric image of an annulus generated in accordance with embodiments of the present disclosure.

FIG. 13 is a volumetric image of the annulus of FIG. 12 showing procedure-specific labels generated in accordance with embodiments of the present disclosure.

FIG. 14 is a flow chart of a method implemented for annuloplasty image guidance in accordance with principles of the present disclosure.

FIG. 15 is a graphic used for performing a left atrial appendage occlusion procedure in accordance with embodiments of the present disclosure.

FIG. 16 is a flow chart of an example left atrial appendage occlusion procedure performed in accordance with embodiments of the present disclosure.

FIG. 17 is a flow chart of an example method performed in accordance with embodiments of the present disclosure.

### DESCRIPTION

The following description of certain examples is in no way intended to limit the disclosure or its applications or uses. In the following detailed description of examples of the present systems and methods, reference is made to the accompanying drawings which form a part hereof, and in which are shown by way of illustration specific examples in which the described systems and methods may be practiced. These examples are described in sufficient detail to enable those skilled in the art to practice the presently disclosed systems and methods, and it is to be understood that other examples may be utilized and that structural and logical changes may be made without departing from the spirit and scope of the present disclosure. Moreover, for the purpose of clarity, detailed descriptions of certain features will not be discussed when they would be apparent to those skilled in the art so as not to obscure the description of the present disclosure. The following detailed description is therefore not to be taken in a limiting sense, and the scope of the present systems and methods is defined only by the appended claims.

Ultrasound systems configured to provide interventional medical procedure image guidance are disclosed, along with associated methods of automating the steps necessary to acquire, display, and adjust specific images in real time for a user, e.g., clinician, performing a procedure. In some examples, volumetric image data of a region of interest (“ROI”) may be obtained by an ultrasound transducer configured to perform 3D imaging. The acquired image data can be transmitted to at least one computer processor equipped with an anatomical recognition processor configured to identify anatomical landmarks within the acquired image data. Based at least in part on the identified landmarks, an image reconstruction processor can generate at least one image of an anatomical feature along at least one imaging plane necessary to perform the interventional procedure. A user interface communicatively coupled with the anatomical recognition processor and the reconstruction processor can display the reconstructed image to the user during the interventional procedure. In this manner, the selection of an interventional procedure via the user interface may dictate which anatomical landmarks are relevant to obtaining the images necessary to perform the procedure. The necessary images are then automatically generated along specific planes and/or from specific volumetric vantage points with little or no manual user manipulation of an ultrasound transducer. A user interface included in the disclosed systems can be used to confirm that the necessary images have been obtained and to adapt to procedural progress and/or deviations for pre-procedural planning specifications. Image data acquired in other, non-ultrasound imaging modalities, e.g., MRI, CT or X-ray, can be used in conjunction with the disclosed systems to further improve the accuracy and efficiency of detecting and displaying images of an anatomical feature along one or more particular imaging planes.

In some embodiments, the reconstructed images can be generated by harvesting the acquired volumetric data for a subset of planar or volumetric data specific to a selected interventional procedure. In addition or alternatively, the reconstructed images can be generated by modifying the ultrasound scan sequence used to acquire the image data in a manner designed to produce optimal anatomical views and enhance image quality. Examples may involve steering imaging planes and/or truncating scan frames and lines such that unnecessary acquisition may be skipped in favor of only the scan line acquisitions necessary for generating optimal, procedurally-relevant views. Examples may also involve auto-cropping, or selectively removing unnecessary imaging data from the initially acquired volumetric data and displaying only the retained data in the form of one or more images. By implementing one or more of the aforementioned image processing measures, increased spatial resolution of the desired images can be attained in a faster, more computationally efficient manner.

The automated generation of image views used to guide interventional procedures marks a significant improvement over preexisting systems that require users to capture the same views manually. Such automation also facilitates fast, effective user navigation between the required views encompassing various organs, e.g., the heart. Decreasing the frequency of manual ultrasound probe manipulation may also enhance the efficiency and accuracy of targeted ultrasound image acquisition, which in turn may translate into shorter procedure times and more reliable ultrasound imaging responsive to real-time user input.

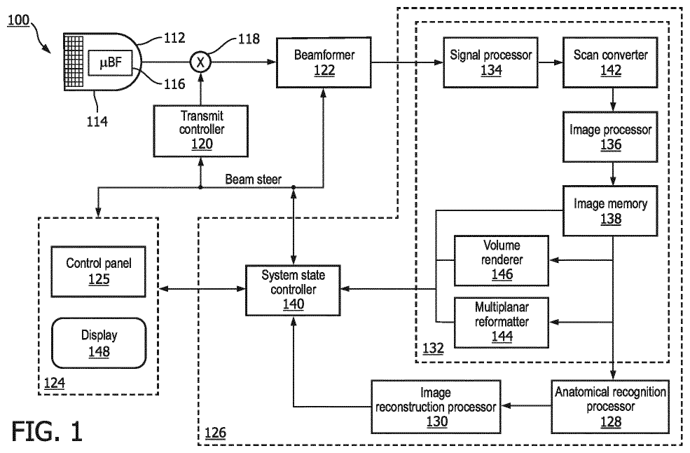

FIG. 1 shows a block diagram of an example ultrasound imaging system 100 constructed in accordance with the principles of the present disclosure. As shown, the system 100 may include a transducer array 114, which may be included in an ultrasound probe 112, for example an external probe or an internal probe such as an intravascular ultrasound (“IVUS”) catheter probe. In other examples, the transducer array 114 may be in the form of a flexible array configured to be conformally applied to a surface of a subject to be imaged (e.g., patient). The transducer array 114 is configured to transmit ultrasound signals (e.g., beams, waves) at a target region, e.g., a chest of a patient, and receive echoes (e.g., received ultrasound signals) responsive to the transmitted ultrasound signals from the target region. A variety of transducer arrays may be used, e.g., linear arrays, curved arrays, or phased arrays. The transducer array 114, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some examples, the transducer array 114 may be coupled to a microbeamformer 116, which may be located in the ultrasound probe 112, and which may control the transmission and reception of signals by the transducer elements in the array 114. In some examples, the microbeamformer 116 may control the transmission and reception of signals by active elements in the array 114 (e.g., an active subset of elements of the array that define the active aperture at any given time).

In some examples, the microbeamformer 116 may be coupled, e.g., by a probe cable or wirelessly, to a transmit/receive (T/R) switch 118, which switches between transmission and reception and protects the main beamformer 122 from high energy transmit signals. In some examples, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface.

The transmission of ultrasonic signals from the transducer array 114 under control of the microbeamformer 116 may be directed by the transmit controller 120, which can be coupled to the T/R switch 118 and the main beamformer 122. The transmit controller 120 may control characteristics of the ultrasound signal waveforms transmitted by the transducer array 114, for example, amplitude, phase, and/or polarity. The transmit controller 120 may also control the direction in which beams are steered. Beams may be steered straight ahead from (orthogonal to) the transducer array 114, or at different angles for a wider field of view. The transmit controller 120 may also be coupled to a user interface 124 configured to receive user input. For example, the user may select whether the transmit controller 120 causes the transducer array 114 to operate in a harmonic imaging mode, fundamental imaging mode, Doppler imaging mode, or a combination of imaging modes (e.g., interleaving different imaging modes). At the user interface 124, the user may also select an interventional procedure to be performed with image guidance provided by the system 100. In some examples, the user interface 124 may include one or more input devices such as a control panel 125, which can include one or more mechanical controls (e.g., buttons, encoders, etc.), touch-sensitive controls (e.g., a trackpad, a touchscreen, or the like), and/or other known input devices (e.g., voice command receivers) responsive to a variety of auditory and/or tactile inputs.

In some examples, the partially beamformed signals produced by the microbeamformer 116 may be coupled to the main beamformer 122 where partially beamformed signals from individual patches of transducer elements may be combined into a fully beamformed signal. In some examples, microbeamformer 116 can be omitted, and the transducer array 114 may be under the control of the main beamformer 122, which can then perform all beamforming of signals. In examples with and without the microbeamformer 116, the beamformed signals of main beamformer 122 are coupled to processing circuitry 126, which may include one or more processors (e.g., an anatomical recognition processor 128, an image reconstruction processor 130, and one or more image generation and processing components 132) configured to produce live, reconstructed ultrasound images from the beamformed signals (e.g., beamformed RF data).

Signal processor 134 may receive the beamformed RF data and process the data in various ways, such as bandpass filtering, decimation, and I and Q component separation. The processing of the beamformed RF data performed by the signal processor 134 may be different based, at least in part, on the particular interventional procedure being performed by the user. Image processor 136 is generally configured to generate image data from the RF data, and may perform additional enhancement such as speckle reduction, signal compounding, spatial and temporal denoising, and contrast and intensity optimization. Radiofrequency data acquired by the ultrasound probe 112 can be processed into various types of image data, non-limiting examples of which may include per-channel data, pre-beamformed data, post-beamformed data, log-detected data, scan converted data, and processed echo data in 2D and/or 3D.

Processed signals output from the signal processor 134 (e.g., I and Q components) may be coupled to additional downstream signal processing circuits for anatomical landmark detection, image reconstruction, and automated user guidance. For example, the signals from the signal processor 134 can be transmitted to the anatomical recognition processor 128 and an image reconstruction processor 130, each of which may be communicatively coupled to the user interface 124.

The anatomical recognition processor 128 can be configured to recognize various anatomical features within a set of image data. Embodiments of the anatomical recognition processor 128 may be configured to recognize such features by referencing and sorting through a large library of stored images. For example, the anatomical recognition processor 128 may comprise a heart recognition processor configured to identify one or more features of a patient's heart, e.g., an atrium, ventricle, valve, annulus, etc., by referencing and sorting through a large library of cardiac images obtained from a sample of patients treated according to a variety of interventional cardiology procedures. The library may be supplemented over time, for instance with new images of additional features not originally included, non-limiting examples of which include images of organs such as the brain, lungs or liver and portions thereof.

The image reconstruction processor 130 can receive the image data output stored or buffered in the image memory 138, use the information gathered by the recognition processor 128 and then generate one or more 2D, planar views of a particular feature of interest along a specific plane, e.g., a planar view and/or at least one cross-sectional view, relevant to an image-guided interventional procedure. Embodiments of the image reconstruction processor 130 may also be configured to use the acquired ultrasound data to generate one or more 3D, volumetric views of a particular feature of interest from a specific vantage point relevant to an image-guided interventional procedure. The image reconstruction processor 130 may reconstruct images in this manner by operating in tandem with one or more additional processors included in the system 100.

Two-dimensional image reconstruction may involve slicing one or more planes present within a set of received volumetric image data and reconstructing one or more new, 2D images along the planes for display on the user interface 124. The image reconstruction processor 130 may automatically produce the desired planar views in response to the selection of a particular interventional procedure and/or in response to a user input received at the user interface 124 before or during an interventional procedure. For example, as described in greater detail below, the image reconstruction processor 130 may be configured to generate a top-down plane view and at least one cross-sectional side view of a mitral valve in response to a user selecting or inputting a valve clip procedure at the user interface 124. The views required to guide a user through a particular interventional procedure may be stored within the system such that at least one procedure-specific view is reconstructed upon user selection of a procedure at the user interface 124 and receipt of sufficient volumetric ultrasound data from the ultrasound probe 112. The reconstruction processor 130 may generate the necessary views sequentially in the order they are needed to perform an interventional procedure or all at once for simultaneous or sequential display at the user interface 124.

In some cases, the image reconstruction processor 130, in conjunction with the anatomical recognition processor 128, may be unable to produce the desired view(s) due to an insufficiency of volumetric data acquired via the probe 112 and supplied to the signal processor 134. This may occur if an anatomical feature, such as the heart, is not fully captured by the user during the initial ultrasound data acquisition process. This situation may prompt the anatomical recognition processor 128 and/or image reconstruction processor 130 to generate an indication to the system state controller 140 that insufficient ultrasound data was captured, and convey the indication to the user interface 124 for display. In some embodiments, the indication may include one or more user instructions for adjusting the position, orientation and/or settings of the ultrasound probe 112 in the manner necessary to acquire volumetric image data sufficient for the anatomical recognition processor 128 to recognize the necessary landmarks and the image reconstruction processor 130 to generate images along the clinically relevant planes.

In some embodiments, the signals produced by the signal processor 134 may be coupled to a scan converter 142 and/or a multiplanar reformatter 144. The scan converter 142 may be configured to arrange the echo signals into the intended geometric format. For instance, data collected by a linear array transducer would represent a rectangle or a trapezoid, whereas the same for a sector probe would represent a sector of a circle.

The multiplanar reformatter 144 can convert echoes received from points in a common plane in a volumetric region of the body into an ultrasonic image of that plane, for example as described in U.S. Pat. No. 6,443,896 (Detmer). The scan converter 142 and multiplanar reformatter 144 may be implemented as one or more processors in some examples.

In embodiments configured to generate a clinically-relevant volumetric subset of image data, a volume renderer 146 configured to generate an image (also referred to as a projection, render, or rendering) of the 3D dataset as viewed from a given reference point, e.g., as described in U.S. Pat. No. 6,530,885 (Entrekin et al.), can be included. The volume renderer 146 may be implemented as one or more processors in some examples. The volume renderer 146 may generate a render, such as a positive render or a negative render, by any known or future known technique such as surface rendering and maximum intensity rendering.

Output (e.g., B-mode images along a particular image plane) from the image processor 136 may be coupled to the local image memory 138 for buffering and/or temporary storage before being displayed on an image display 148 through the system state controller 140.

The system state controller 140 may generate graphic overlays for display with the images. These graphic overlays can contain, e.g., standard identifying information such as patient name, date and time of the image, imaging parameters, and the like. For these purposes, the system state controller 140 may be configured to receive input from the user interface 124, such as a typed patient name or other annotations. The graphic overlays can also contain information specific to a selected procedure, e.g., one or more image plane labels, anchor points, indications or alerts received from other components of the system 100, or selectable user instructions for obtaining an image along one or more image planes. For these purposes, the system state controller 140 may be configured to receive input from the user interface 124 encompassing or related to an interventional procedure selection and/or confirmation that one or more interventional steps have been successfully performed by the user, which may prompt the system 100 to acquire an additional image pursuant to a next step of the procedure. The user interface 124 can also be coupled to the multiplanar reformatter 144 for selection and control of a display of multiple multiplanar reformatted (MPR) images.

The system 100 may include local memory 138. Local memory 138 may be implemented as any suitable non-transitory computer readable medium (e.g., flash drive, disk drive). Local memory 138 may store data generated by the system 100 including images, executable instructions, inputs provided by a user via the user interface 124, or any other information necessary for the operation of the system 100.

User interface 124 may include a display 148 and a control panel 125. The display 148 may include a display device implemented using a variety of known display technologies, such as LCD, LED, OLED, or plasma display technology. In some examples, display 148 may comprise multiple displays. The control panel 125 may be configured to receive user inputs (e.g., selection of interventional procedures, imaging modes, selection of regions of interest, image adjustments). The control panel 125 may include one or more hard controls (e.g., buttons, knobs, dials, encoders, mouse, trackball or others). In some examples, the control panel 125 may additionally or alternatively include soft controls (e.g., GUI control elements or simply, GUI controls) provided on a touch sensitive display, which may overlap with display 148, such that a user can interact directly with the images shown on the display 148, for example by touch-selecting certain anatomical features for enhancement and/or indicating the position or orientation of interventional devices to be implanted with such anatomical features. In some examples, the display 148 may be a touch-sensitive display that includes one or more soft controls of the control panel 125. The user interface 124 may also be used to adjust various parameters of image acquisition, generation, and/or display. For example, a user may adjust the power, imaging mode, level of gain, dynamic range, turn on and off spatial compounding, and/or level of smoothing. In some embodiments, the user-adjustable settings may affect the imaging mode.

In some embodiments, various components shown in FIG. 1 may be combined. For instance, the anatomical recognition processor 128, image reconstruction processor 130, image processor 136 and/or system state controller 140 may be implemented as a single processor. In some examples, various components shown in FIG. 1 may be implemented as separate components. For example, signal processor 134 may be implemented in the form of separate signal processors for each imaging mode (e.g., B-mode, color). In some examples, one or more of the various processors shown in FIG. 1 may be implemented by general purpose processors and/or microprocessors configured to perform the specified tasks described herein. In some examples, one or more of the various processors may be implemented as application specific circuits. In some examples, one or more of the various processors (e.g., image processor 136) may be implemented with one or more graphical processing units (GPUs). In other examples, one or more of the various processors (e.g., signal processor 134) may be implemented with one or more field programmable gate arrays (FPGAs).

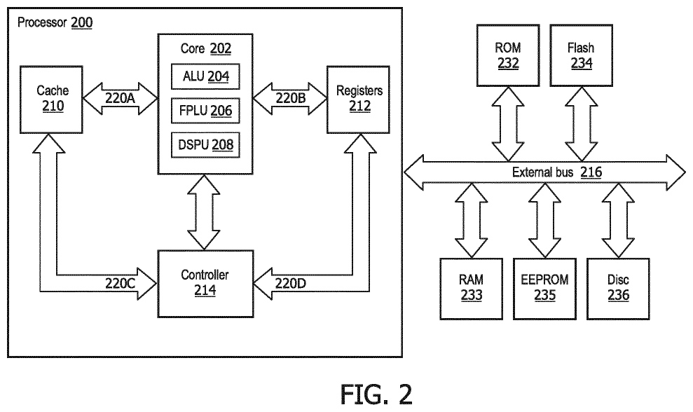

FIG. 2 is a block diagram illustrating an example processor 200 according to principles of the present disclosure. Processor 200 may be used to implement one or more processors described herein, for example, the image processor 136 shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 804. In some examples, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some examples the registers 212 may be implemented using static memory. The register may provide data, instructions and addresses to the core 202.

In some examples, processor 200 may include one or more levels of cache memory 210 communicatively coupled to the core 202. The cache memory 210 may provide computer-readable instructions to the core 202 for execution. The cache memory 210 may provide data for processing by the core 202. In some examples, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., user interface 124) and/or outputs from the processor 200 to other processors and/or components included in the system (e.g., display 148). Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache memory 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache 210, and/or register 212. The bus 216 may be coupled to one or more components of the system, such as display 148 and control panel 125 mentioned previously.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 233. RAM 233 may be a static RANI, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some examples, the external memories may be included in a system, such as ultrasound imaging system 100 shown in FIG. 1, for example local memory 138.

Embodiments of the systems disclosed herein may be configured to selectively acquire only the ultrasound image data necessary to obtain an enhanced image of a targeted anatomical feature from one or more clinically-relevant views determined based on an understanding of the exact needs of a user performing an interventional procedure. Once the anatomical feature is identified within the acquired image data, the system can be configured to “zero-in” on that feature and in doing so, cease obtaining and/or processing unnecessary image data from extraneous anatomical regions. In this manner, the system can increase the processing speed of the system and maintain a volume frame rate while also increasing the resolution and overall quality of the final, reconstructed images.

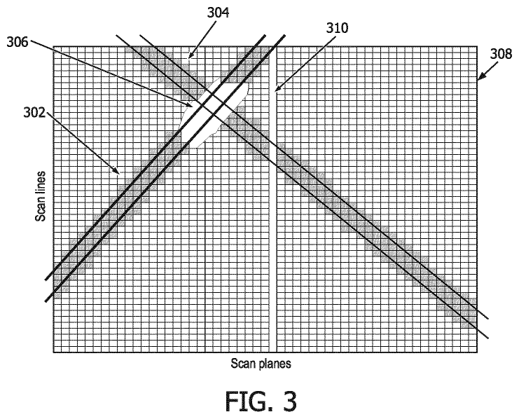

The improved efficiency of the system is portrayed in FIG. 3, which depicts a first set of scan lines 302 and a second set of scan lines 304 necessary for producing cross-sectional 2D images of a targeted anatomical feature 306 through clinically-relevant image planes. To acquire volumetric image data from a region of interest 308 containing the anatomical feature 306, an ultrasound transducer may traditionally sweep through a series of scan lines and scan planes, such as scan plane 310. According to embodiments disclosed herein, however, the ultrasound transducer may be configured to selectively acquire image data only along the scan lines and scan planes necessary to obtain the image data required to reconstruct the clinically-relevant views of the object 306. By scanning only the relevant areas of the interrogated volume, e.g., the areas targeted by scan lines 302 and 304, the scanline and scan plane density can be increased significantly without reducing the volumetric image data acquisition rate. One result of this tradeoff may be enhanced spatial resolution of the ultrasound images reconstructed along the clinically-relevant planes. In addition to optimizing image resolution and quality, this functionality may improve the overall performance of the system by reducing computational load and improving processing speed and efficiency.

In accordance with one embodiment of image quality enhancement, the anatomical recognition processor may determine the optimal scanning depth necessary for reconstructing an image along a desired plane. For example, if a user is seeking a top-down plane view of a mitral valve, the user may not be interested in the anatomical features present far below the valve leaflets. In this situation, the anatomical recognition processor can determine the maximum scanning depth necessary to capture the valve leaflets, and signal the ultrasound acquisition components to adjust the pulse repetition frequency accordingly. The features of interest can thus be prioritized by selecting high-priority on a display shown on the user interface. This selection may increase the scanline and scan plane densities targeting only the selected features, such that an acceptable volume rate is maintained. In some embodiments, a controller (e.g., transmit controller 120) may be configured to cause the image data acquisition components of the system (e.g., ultrasound probe 112) to cease transmitting ultrasound signals except for the signals required to generate the planar ultrasound image along the relevant image plane.

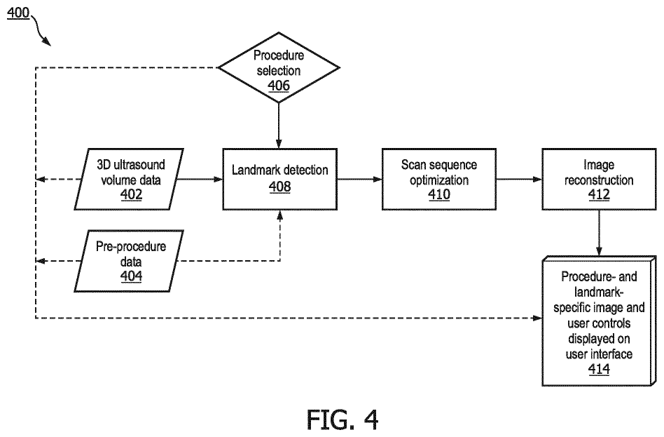

FIG. 4 is a flow chart showing several method steps 400 performed by embodiments of the disclosed systems, along with several notable inputs received by the disclosed systems. As shown, inputs received in accordance with various embodiments may include 3D ultrasound volume data 402, pre-procedure data 404, and a procedure selection 406, one or more of which may be input by a user or received from a database storing patient- and/or procedure-specific information. Based on these inputs, a system can perform anatomical landmark detection 408, optimize an image scan sequence 410, and reconstruct at least one ultrasound image 412 specific to both the selected procedure and the anatomical landmark(s) detected for display on a user interface 414. As further shown, the 3D ultrasound volume data 402, pre-procedure data 404, and procedure selection 406 can also cause the user interface 414 to display user controls specific to such inputs. In various examples, a system implementing one or more of the method steps 400 can be configured to generate and display one or more specifically oriented, planar images of at least one anatomical feature relevant to performing an interventional medical procedure based on the received inputs 402, 404 and 406 and landmark detection 408. Non-limiting examples of such anatomical features may include one or more heart valves or leaflets, which can be selected by a user depending on the particular procedure being performed, the particular medical instrumentation used, and/or the particular medical devices being implanted. Orientations of the anatomical features, which can also be selected by the user, may include top-down plane views and/or cross-sectional views, for example. The user performing the interventional procedure may then rely on the planar images generated by the system to perform the procedure, which may involve inserting a medical device through, within, or in close proximity to the anatomical feature. The planar images may be displayed sequentially to coincide with the steps involved in the selected procedure, and in some examples, the user may confirm, via the user interface 414, that each step has been successfully performed before a next planar image is generated and displayed. Embodiments may also be configured to display one or more graphics, e.g., medical device landing zones, insertion locations, and/or anchorage points, on the user interface 414 to further guide the user during the interventional procedure.

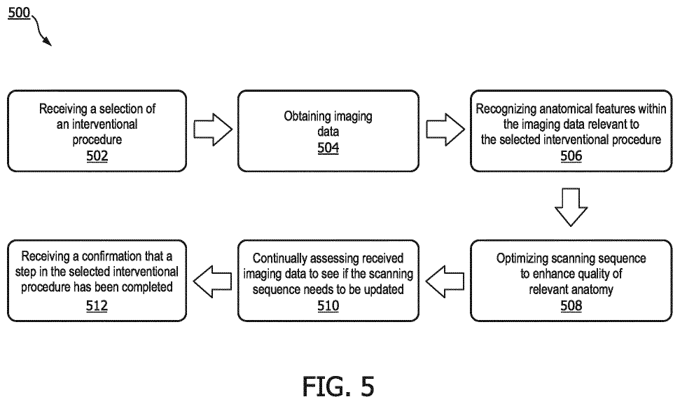

FIG. 5 is a flow chart of an example method 500 implemented in accordance with principles of the present disclosure. In some examples, the method 500 may be performed by an ultrasound imaging system, such as ultrasound imaging system 100.

At block 502, the system (e.g., ultrasound imaging system 100) receives a user selection of an interventional medical procedure, non-limiting examples of which may include a cardiovascular valve clip procedure, an annuloplasty procedure, or an appendage occlusion procedure, just to name a few. The procedure may be input by the user manually in free-text form, located via a search tool, and/or selected from a pre-set list of procedure options, e.g., organized in a drop-down menu or buttons displayed on a user interface. The user interface may also be configured to display procedure-specific graphics and controls. For example, the user interface may be configured to display a graphic enabling user selection of one or more specific valves or leaflets targeted by the user. The user interface may also be configured to display selectable orientation options for planar views of the targeted anatomical feature, along with one or more graphics highlighting a medical device implantation zone or location, which may be overlaid on a current image. In some examples, the graphics indicating medical device placement options can be adjusted by the user before or during the interventional procedure. For instance, the user interface may display a landing zone for a medical implant along a perimeter of the annulus. The user may then adjust the location of the landing zone based on various factors, e.g., newly-discovered patient-specific anatomy and/or user experience. Such an adjustment may be input at the user interface, such that the user-modified landing zone is then displayed.

At block 504, the system obtains and receives imaging data via an ultrasound transducer interrogating a volume of interest, e.g., a chest region of a patient. As disclosed herein, the imaging data may comprise 3D ultrasound volume data.

At block 506, the system may automatically recognize and/or identify anatomical features within the imaging data relevant to the selected interventional procedure. If not, the system may cause a graphic or other alert to be displayed on the user interface indicating that the required anatomical features were not captured in the initial scan sequence. In some examples, the system may also generate and display an instruction for adjusting the image acquisition device, e.g., ultrasound probe, in the manner necessary to acquire the necessary image data.

At block 508, the system may use information regarding where the relevant anatomical feature occurs relative to the transducer and optimize the scanning sequence accordingly to enhance the image quality of the relevant anatomy. This step may also improve the efficiency and speed of image acquisition and display.

At block 510, the system may continue to assess the suitability of the optimized scanning sequence using incoming imaging data to determine if the scanning sequence needs to be updated. This may include occasionally acquiring imaging data using an unoptimized scanning sequence to resample the scanning area.

At block 512, the system can receive confirmation from the user, for example via manual or audible input received at a user interface, that a specific interventional step has been successfully completed. The user can then indicate the next desired step, which may re-start the process.

### EXAMPLES

The following examples should be construed as non-limiting illustrations of how the disclosed systems and methods may be implemented for specific interventional medical procedures. Accordingly, the procedures, anatomical features, medical devices, and image graphics, etc. referenced below are described for illustrative purposes only, and as such, different procedures, anatomical features, medical devices, and image graphics, etc. may be selected and/or displayed in other examples. As set forth below, each procedure may be associated with different relevant landmarks and image views required for user guidance, each of which may be accompanied by changes in the display produced by the user interface.

### Example 1—Valve Clip Procedures

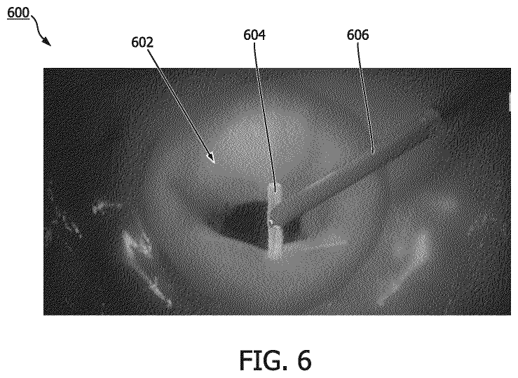

Embodiments of the disclosed systems can be configured to guide a user through various cardiovascular valve clip procedures, which often involve joining two or more valve leaflets or commissures with an implantable clip device. Clip procedures of the mitral or tricuspid valves, for example, typically require the acquisition and display of at least two different views of the treatment site to provide the user with the image guidance necessary to accurately position the clip. In mitral valve clip procedures, the first view may be a “top-down” plane view 600 of the mitral valve 602, which is shown in FIG. 6. This plane view 600 may be important for confirming the location of a valve clip 604 relative to the targeted valve leaflets and/or commissures upon placement of the clip 604 by an interventional device, which comprises a catheter 606 in the illustrated example. It may also be important to confirm that the orientation of the clip 604 is aligned perpendicular to the coaptation of interest. In the particular example shown in FIG. 6, the mitral valve leaflets will be clipped together at the A2 and P2 commissures, which can be confirmed using the top-down plane view 600. Supplied only with this view, however, it may still be difficult for the clinician to determine if the trajectory of the clip 604 is correct.

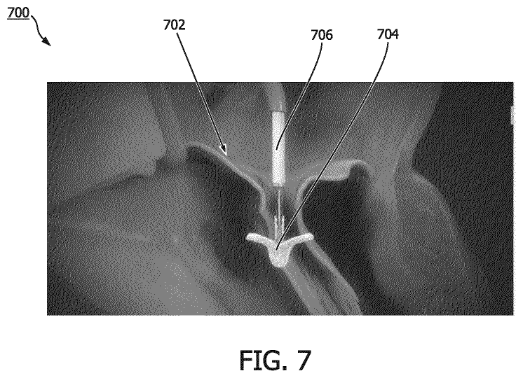

Accordingly, the second important view typically comprises a cross-sectional view 700 of the mitral valve 702, as shown in FIG. 7. Obtaining this view 700 may be necessary to ensure the trajectory of the clip 704 and catheter 706 is perpendicular to the mitral leaflet surface plane when the valve is closed. If the imaging plane is not correctly aligned with the trajectory of the interventional devices during the insertion process, the clip 704 could be placed in the wrong position, where it may inadvertently snag the valve commissures at an angle causing stress to the valve leaflets during each cardiac cycle. In addition, inadequate imaging that leads to improper clip placement may prevent the clip 704 from deploying due to interference by chordae, which may be hidden out of the imaging plane.

Obtaining a clear image along the cross-sectional plane of the valve 702, as illustrated in FIG. 7, can be difficult via manual ultrasound transducer manipulation because the center of the transducer aperture must be positioned precisely above the desired trajectory of the clip to obtain the necessary images. Positioning the transducer aperture in this manner can be a time-consuming process, which may be even more difficult to achieve for tricuspid valve clip procedures. Some preexisting technologies, such as systems configured to reconstruct 2D images from 3D datasets, may remove the constraint of precise relative transducer location, but still require the user to obtain the image planes manually, a task which usually needs to be redone every time the transducer moves relative to the anatomy.

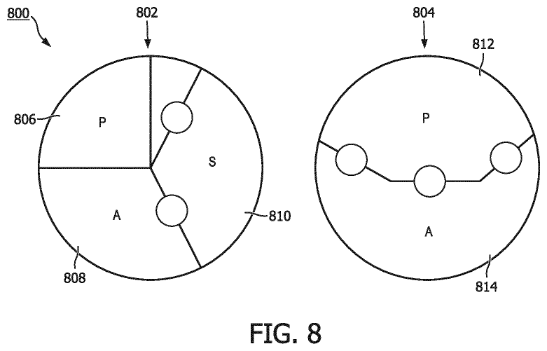

To reduce user difficulty, minimize error, and shorten the procedural duration, embodiments of the systems disclosed herein can include an adaptable user interface configured to display options to the user for valve selection and clip placement before the interventional procedure even begins. For example, FIG. 8 shows an example display 800 that includes a tricuspid valve graphic 802 and a mitral valve graphic 804. A user can select, for example via touchscreen, either of the displayed valve graphics depending on which interventional procedure is being performed. The discrete valve leaflets are also depicted on each valve graphic for user selection. In particular, the tricuspid valve graphic 802 includes a selectable posterior leaflet 806, a selectable anterior leaflet 808, and a selectable septal leaflet 810. The mitral valve graphic 804 includes a selectable posterior leaflet 812 and a selectable anterior leaflet 814.

Selection of a valve and/or at least one leaflet may initiate volumetric ultrasound image acquisition of the portion of the body encompassing the selected valve, which in this example would include a portion of a patient's chest. If the valve of interest is not captured within the volume scanned by the ultrasound transducer, one or more processors, e.g., the anatomical recognition processor 128 and/or an image reconstruction processor 130 shown in FIG. 1, may generate a signal for display on the user interface, thereby prompting the user to adjust the transducer position and/or orientation.

Based on the anatomical landmark(s) identified within the volumetric data by the anatomical recognition processor, user selection of the valve and/or leaflet(s) of interest may prompt the anatomical recognition processor to also determine the specific orientation of the annulus plane, the leaflet/commissure surface plane, and the coaptation plane. The orientation of the leaflet surface plane may be defined by the plane created by the surface of the leaflets that will be joined pursuant to the procedure, and for mitral valves, the surface of the commissure surface plane may be defined by the surface of the commissures that will be joined. The coaptation plane may be defined as the plane formed by the joined portion of the coaptation. Upon identifying the necessary anatomical landmarks and at least one of the aforementioned orientations, the anatomical recognition processor may automatically cease processing image data for the remainder of the entire heart to reduce the computational processing output.

The image reconstruction processor may then generate a top-down plane view showing the valve annulus plane using the acquired volumetric data and the identified landmarks. From this plane, the user can select, at the user interface, a landing zone for placing the valve clip. The image reconstruction processor may then generate at least one additional image along a plane orthogonal to the top-down plane view, such as the cross-sectional view shown in FIG. 7. In some examples, the reconstruction processor may generate two cross-sectional views rotated 90° with respect to each other, such that the rotational axis is perpendicular to the leaflet/commissure surface plane and centered on the landing zone selected by the user.

After supplying the user with the necessary views for inserting the interventional device and placing the valve clip along the selected landing zone, the anatomical recognition processor may or may not continue to monitor the valve orientation along one or more of the aforementioned planes. Instead, the anatomical recognition processor may be ECG-gated such that anatomical recognition and/or image reconstruction is performed only once per cardiac cycle during diastole when the mitral or tricuspid valves are closed. Embodiments can also be configured to accommodate on-demand image analysis and reconstruction prompted by user input received at the interface, for example if the patient and/or transducer moves during the procedure.

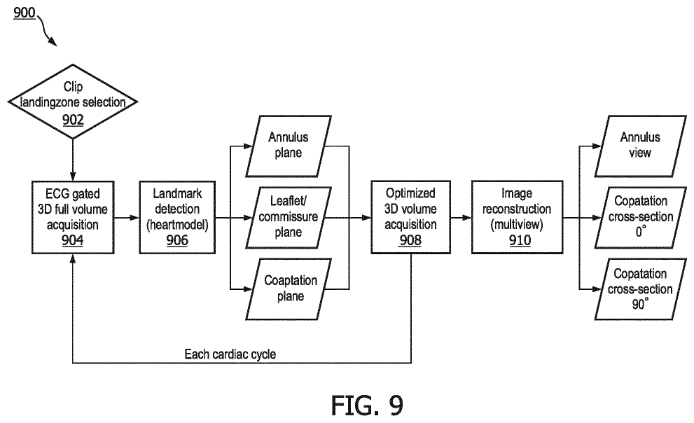

A flow chart of an example valve clip procedure 900 performed in accordance with at least one embodiment described herein is represented in FIG. 9. As shown, the procedure may begin at step 902 with the selection of a landing zone for a valve clip. This selection may be received from a user, for example via user interface 124. In some examples, the selection of a landing zone may be received via user interaction with an ultrasound image displayed on user interface 124. For example, the user interface may comprise a touch screen configured to display a landing zone graphic responsive to the user input, showing the location, size and/or anchor points of the landing zone. The procedure may continue at step 904 with ECG-gated full-volume acquisition (e.g., via ultrasound probe 112), which may then trigger automatic detection of anatomical landmarks at step 906 (e.g., via anatomical recognition processor 128). The landmarks may include and/or enable identification of the annulus plane, leaflet/commissure plane, and coaptation plane. Volumetric image data acquisition may then be optimized at step 908, for example by obtaining only the image data necessary to view the annulus plane, leaflet/commissure plane and/or coaptation plane, after which the image reconstruction processor may, at step 910, automatically generate, in real time or at regular intervals, at least one view of the valve necessary for precise valve clip placement. The necessary views may include an annulus view, a coaptation cross-section at and a coaptation cross-section at 90°.

### Example 2—Annuloplasty Procedures

Embodiments of the disclosed systems can be configured for guiding annuloplasty procedures. For example, the Cardioband annuloplasty system (Edwards Lifesciences, Irvine, CA) is a commercial valve reconstruction system featuring an implant (the Cardioband) deployed and anchored along the annulus. Once anchored in the proper position, the implant is contracted to remodel the annulus and reduce regurgitation. A combination of fluoroscopic and transesophageal echocardiography (“TEE”) image guidance is typically used to deploy and adjust the implant until properly seated around the annulus. One of the primary challenges in the deployment of this device is ensuring that all of the anchors used to secure the implant to the annulus, which may total 16 or more anchors, are placed correctly.

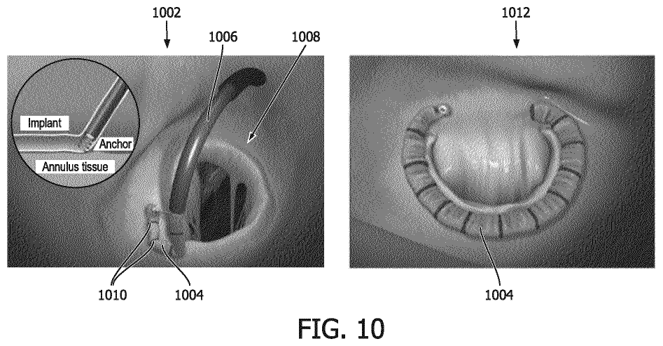

The anchoring in-progress image 1002 of FIG. 10 shows the implant 1004 being unraveled from the end of a catheter 1006 at its targeted location around the annulus 1008. Each anchor 1010 used to secure the implant 1004 can be placed individually as the implant is gradually deployed from the catheter 1006 by inserting each anchor directly into the annulus tissue. As shown in the post-implantation image 1012, the secured implant may surround a portion of the annulus perimeter. Critical to proper anchor placement is the trajectory of each anchor relative to the implant, the valve annulus, and other surrounding anatomy. For example, the trajectory of each anchor must be angled such that it can attach the implant to the annulus against the force vectors that are applied to the annulus throughout each cardiac cycle. Moreover, each anchor must avoid hitting any critical anatomy surrounding the annulus, such as the aortic root and coronaries.

To minimize the likelihood of ineffective and/or unsafe implant deployment, the automated image guidance provided by systems disclosed herein can be configured to automatically generate a top-down plane view of the implant landing zone around an annulus, thereby improving anchor placement planning. The disclosed systems can also be configured to automatically create cross-sectional image planes for each anchor, thereby enabling precise anchor placement on an anchor-by-anchor basis.

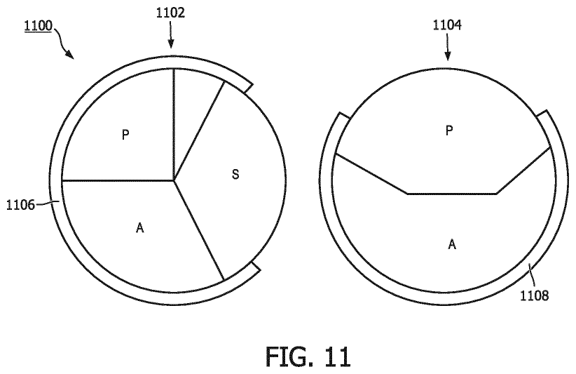

In operation, a user can select a valve of interest at the user interface (e.g., user interface 124) by interacting with a graphical display 1100 similar to that shown in FIG. 11, which includes a tricuspid valve graphic 1102 and a mitral valve graphic 1104. A user can select, for example via touchscreen, either of the displayed valve graphics depending on which valve annulus is being targeted, i.e., the tricuspid annulus 1106 or the mitral annulus 1108.

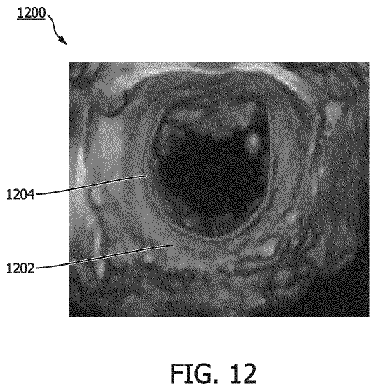

Before, during or after selection of a particular valve annulus, the system can acquire volumetric image data using an ultrasound transducer to produce a volumetric image 1200, as shown for example in FIG. 12. Using the information selected by the user via the user interface and the volumetric image data acquired by the ultrasound transducer, a landing zone 1202 can be rendered onto the 3D image 1200, along with an outline of the leaflet hinge 1204. The displayed landing zone 1202 can be selected during the pre-procedure planning stage and then rendered onto a live ultrasound image during the annuloplasty procedure, where it may be adjusted in real time by the user interacting with the user interface.

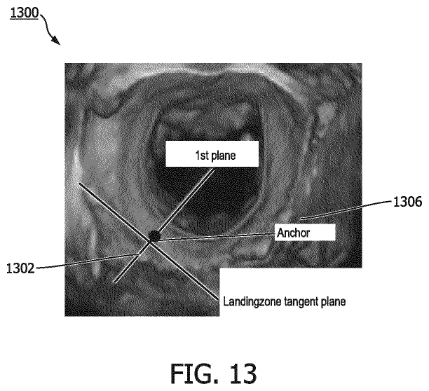

The user can also select specific anchor points within the landing zone 1202, for example by simply tapping the user interface at the targeted locations within the displayed landing zone. During anchor placement, the user can indicate, again at the user interface, which anchor is about to be placed, which may then prompt the system to automatically create two or more cross-sectional views at the anchor site. For example, as shown in the volumetric image 1300 of FIG. 13, a first cross-sectional plane 1302 and a second, perpendicular cross-sectional plane 1304 may intersect at the anchor point 1306. This process can be repeated for each anchor to ensure that each anchor is secured at a precise location (e.g., via the top-down plane view) and angle (e.g., via one or more cross-sectional views).

The anatomical recognition processor may, again, cease monitoring the plane orientation continuously, and may instead operate at an interval, for example only once per cardiac cycle, for improved efficiency. The system may also update the user interface display to show the relevant planes for each anchor in real time as they are placed according to a pre-planned procedural protocol. In some examples, an optimized relevant-plane image may be captured in real time, and the system can be configured to re-determine the optimal image plane periodically, such as once per cardiac cycle. In some embodiments, the system can respond to on-the-fly user input, for example if an anchor position needs to be modified during an operation. This functionality may be implemented in tandem with stored procedural specifications or a previously-input, customized procedure plan entered by the user. Accordingly, the procedural planning information can be integrated with the image data acquired in real time. Still further, the system can display the image planes necessary for placement of a next anchor after a given anchor is successfully deployed, thereby providing a guide for the user to follow. This display can also be adapted in the event of a deviation between the pre-planned anchor points and the actual anchor points.

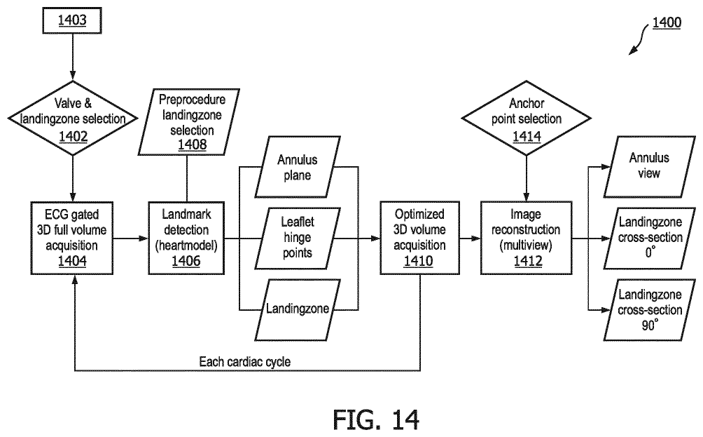

A flow chart of an example annuloplasty procedure 1400 performed in accordance with at least one embodiment described herein is represented in FIG. 14. As shown, the procedure may begin at step 1402 with the selection, via user input 1403 received at a user interface (e.g., user interface 124) of a valve and landing zone for an annulus implant. The procedure may continue at step 1404 with ECG-gated full-volume ultrasound acquisition performed using an ultrasound probe (e.g., probe 112) under the direction of a controller (e.g., transmit controller 120) communicatively coupled with at least one beamformer (e.g., microbeamformer 116 and/or beamformer 122) and a signal processor (e.g., processor 134), for example, which may then trigger automatic detection of anatomical landmarks at step 1406 (e.g., via anatomical recognition processor 128), a step that may also be impacted by pre-procedural landing zone selections 1408. The landmarks identified at step 1406 may include the annulus plane, leaflet hinge points, and landing zone. Volumetric image data acquisition may then be optimized at step 1410, for example by directing (e.g., via transmit controller 120) the ultrasound probe (e.g., probe 112) to cease acquiring image data except for the image data required to generate an image along the relevant plane(s), after which the image reconstruction processor (e.g., processor 130) may, at step 1412, automatically reconstruct at least one view of the annulus necessary for implant deployment. Using the view generated during step 1412 and displayed on a user interface, the system may prompt the user, e.g., via one or more graphics displayed on the user interface, to select the necessary implant anchor points at step 1414, after which the system may generate and display a top-down annulus view necessary for anchor positioning, along with perpendicular landing zone cross-sectional views necessary to ensure that each anchor is secured at the proper angle.

### Example 3—Left Atrial Appendage Occlusion Procedures

Embodiments of the disclosed systems can be configured for guiding left atrial appendage (“LAA”) occlusion procedures. The objective of LAA occlusion procedures is to cut off the appendage from any hemodynamic interaction with the rest of the atrium. During most LAA occlusion procedures, three critical pieces of information are typically needed: confirmation of no thrombus in appendage, confirmation of no appendage side lobes, and the minimum and maximum diameters of the atrial neck.

To assess the existence of thrombi or appendage side lobes, a sufficient 3D image is a prerequisite. Since the LAA is located near the right edge of a 3D image obtained from a midesophageal view, users must manually manipulate the transducer tip using a standard 90° field of view (“FOV”) image in order to adequately capture the entire LAA. Ultrasound imaging modes may benefit from allowing a higher maximum FOV, e.g., about 120°. Instead of scanning with the maximum FOV, the user can thus maintain the 90° but swing the FOV out to the right in order to center the LAA in the image while maintaining a constant frame rate. This may allow the user to get a full picture of the LAA in three dimensions.

During device deployment, sufficient image guidance is typically provided by generating multiple 2D images of the LAA cross-section. For example, a current standard protocol involves assessing the LAA at a 0°, 45°, 90°, and 135° plane rotation angles. Another useful set of views typically includes LAA cross-sectional planes that provide the maximum neck diameter and minimum neck diameter.

Part of the challenge in providing these views from the user's perspective is a consequence of the location of the LAA relative to the transducer tip. Unless the LAA is directly in front of the transducer tip, any plane rotation angle adjustment will foreshorten the cross-section of the LAA, forcing the user to manipulate the transducer each time a new rotation angle is required. Under these circumstances, reliably finding a cross-sectional view of the LAA which maximizes or minimizes the neck diameter can be extremely difficult.

In view of these challenges, embodiments of the systems disclosed herein can be configured to receive a user input selecting the LAA occlusion procedure, for example via a drop-down menu displayed on the user interface. After acquiring 3D ultrasound data that includes the left atrium, the anatomical recognition processor may identify the plane of the ostium. The ostium diameters may also be measured to determine the minimum and maximum. These measurements can be ECG-gated to occur at the end of systole. The neck diameter measurements may also be acquired automatically.

The image reconstruction processor can then process the received image data using the identified landmarks to generate 2D image slices relevant to the LAA occlusion procedure. Such views may include the max-ostium plane perpendicular to the ostium plane, and the min-ostium plane, which is perpendicular to the ostium plane.

Using these views, the user can manually define where the neck is and the system can provide the minimum and maximum diameters thereof. The system can provide a mechanism for the user to manually adjust the orientation of the neck plane to fine tune the measurement. For the guidance portion, as mentioned above, the standard protocol often requires 2D imaging plane rotation angles of 0°, 45°, 90°, and 135° where the rotation occurs on the normal axis of the transducer face. These imaging planes may not be ideal, however. The same rotation angles can be provided according to the systems disclosed herein, but with the rotation occurring about the axis centered on the ostium. As long as the entire LAA is captured in the 3D volume, the system may be configured to generate these rotational angle views that conform to the preexisting protocol, independent of actual transducer orientation. Since the transducer orientation may not provide a useful reference point, the system may allow the user to specify what the 0° point is via the user interface. For example, the 0° point may be defined as the transducer's “natural” non-rotated plane, the max-ostium plane, or the min-ostium plane.

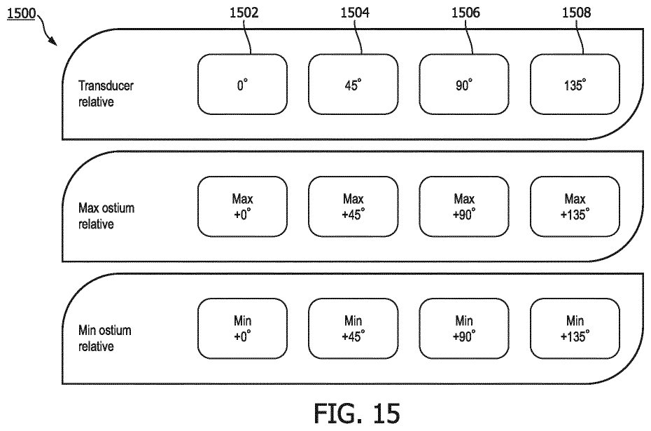

In some embodiments, all relevant views may not be displayed simultaneously on one monitor. The user can instead select a particular view for display via the user interface. The selectable options are depicted in FIG. 15, which depicts an embodiment of a display (e.g., display 148) shown on a user interface (e.g., user interface 124). As an example, the user may select a desired view at interface selection screen 1500, where the user may select a 0 degree transducer angle button 1502 to obtain a desired view, a 45 degree transducer angle button 1504, a 90 degree transducer angle button 1506, and a 135 degree transducer angle button 1508.

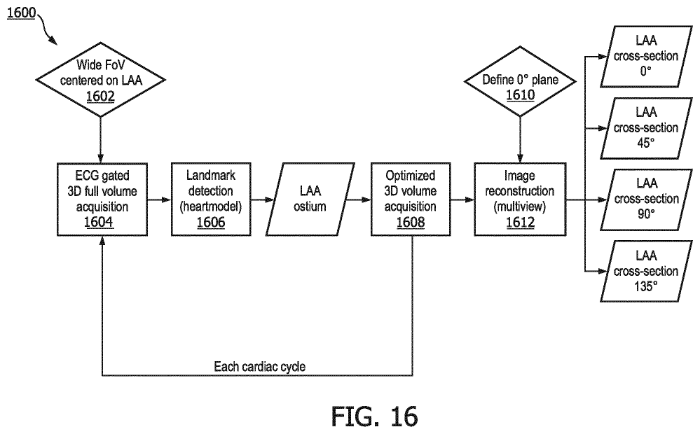

A flow chart of an example LAA occlusion procedure 1600 performed in accordance with at least one embodiment described herein is represented in FIG. 16. As shown, the procedure may begin at step 1602 with the centering of a wide FOV over the LAA. The procedure may continue at step 1604 with ECG-gated full-volume acquisition, performed for example using an ultrasound probe (e.g., probe 112) under the direction of a controller (e.g., transmit controller 120) communicatively coupled with at least one beamformer (e.g., microbeamformer 116 and/or beamformer 122) and at least one signal processor (e.g., processor 134), which may then trigger automatic detection of anatomical landmarks at step 1606 (performed for example via anatomical recognition processor 128). The landmarks may include and/or enable identification of an LAA ostium. Volumetric image data acquisition may then be optimized at step 1608, for example by directing (e.g., via transmit controller 120) the ultrasound probe (e.g., probe 112) to cease acquiring image data except for the image data required to generate an image along the relevant plane(s) responsive to the user selection of one or more view angles), after which the image reconstruction processor (e.g., processor 130) may, after the 0° plane is defined at step 1610, automatically generate at least one view of the ostium at step 1612 for display on a user interface. The necessary views may include an LAA cross-section at 0°, an LAA cross-section at 45°, an LAA cross-section at 90°, and/or an LAA cross-section at 135°, which may be displayed simultaneously or sequentially as the procedure is being performed. In some examples, the user may input at the user interface a confirmation that one or more interventional steps have been performed, which may cause the display on the user interface, under the control of one or more underlying processing components (e.g., image processor 136), to display a subsequent planar image corresponding to the next step in the interventional procedure.

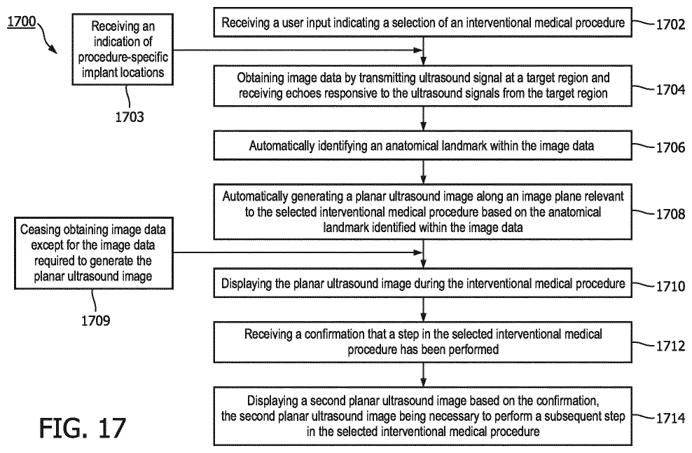

FIG. 17 is a flow chart of an example method 1700 implemented in accordance with various embodiments described herein. The method 1700 may be performed by an ultrasound imaging system, such as ultrasound imaging system 100. The steps of method 1700 may be performed chronologically in the order depicted, or in any order. Accordingly, the particular sequence of steps shown in FIG. 17 should not be construed as limiting.

At block 1702, the system receives a user input indicating a selection of an interventional medical procedure. In some embodiments, the procedure may include a cardiovascular clip procedure, an annuloplasty procedure, or a left atrial appendage occlusion procedure. As shown in block 1703, the system may also receive an indication of procedure-specific implant locations. In some examples, the system may receive the indication of procedure-specific implant locations after displaying a planar ultrasound image (see block 1710), and/or after displaying a second planar ultrasound image (see block 1714). At block 1704, the system obtains image data by transmitting ultrasound signals at a target region and receiving echoes responsive to the ultrasound signals from the target region. The system then, at block 1706, automatically identifies an anatomical landmark within the image data. At block 1708, the system automatically generates a planar ultrasound image along an image plane relevant to the selected interventional medical procedure based on the anatomical landmark identified within the image data. The planar ultrasound image may be generated in real time or in an interval or a period basis, and as shown in block 1709, the system may cease obtaining image data except for the image data required to generate the planar ultrasound image. At block 1710, the system displays the planar ultrasound image during the interventional medical procedure. As noted in block 1712, the system can also receive a confirmation, for example via user input, that a step in the selected interventional medical procedure has been performed, and as shown in block 1714, display a second planar ultrasound image based on the confirmation, the second planar ultrasound image being necessary to perform a subsequent step in the selected interventional medical procedure.

In various examples where components, systems and/or methods are implemented using a programmable device, such as a computer-based system or programmable logic, it should be appreciated that the above-described systems and methods can be implemented using any of various known or later developed programming languages, such as “C”, “C++”, “C#”, Java, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software, and/or firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instructions to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, examples or processes described herein may be combined with one or more other examples, examples and/or processes or be separated and/or performed amongst separate devices or device portions in accordance with the present systems, devices and methods.

Finally, the above-discussion is intended to be merely illustrative of the present systems and methods and should not be construed as limiting the appended claims to any particular example or group of examples. Thus, while the present system has been described in particular detail with reference to exemplary examples, it should also be appreciated that numerous modifications and alternative examples may be devised by those having ordinary skill in the art without departing from the broader and intended spirit and scope of the present systems and methods as set forth in the claims that follow. Accordingly, the specification and drawings are to be regarded in an illustrative manner and are not intended to limit the scope of the appended claims.

1. An ultrasound imaging system comprising:
a user interface configured to receive a user input indicating a selection of an interventional medical procedure;
an ultrasound probe configured to transmit ultrasound signals at a target region and receive echoes responsive to the ultrasound signals and generate radio frequency data corresponding to the echoes;
an image processor configured to generate image data from the RF data;
an anatomical recognition processor configured to receive the image data and identify an anatomical landmark within the image data; and
an image reconstruction processor configured to generate a planar ultrasound image along an image plane relevant to the selected interventional medical procedure based on the anatomical landmark identified within the image data,
wherein the user interface is configured to display the planar ultrasound image during the interventional medical procedure.

2. The ultrasound imaging system of claim 1, wherein the interventional medical procedure comprises a cardiovascular valve clip procedure, an annuloplasty procedure, or a left atrial appendage occlusion procedure.

3. The ultrasound imaging system of claim 1, wherein the image data comprises 3D data acquired via a volumetric imaging mode.

4. The ultrasound imaging system of claim 1, wherein the user interface is further configured to receive an indication of procedure-specific implant locations.

5. The ultrasound imaging system of claim 1, wherein the image reconstruction processor is configured to generate at least two planar ultrasound images along at least two image planes relevant to the selected interventional medical procedure based on the anatomical landmark identified within the image data and interventional devices required to perform the interventional medical procedure.

6. The ultrasound imaging system of claim 5, wherein the user interface is configured to display the images sequentially as the interventional medical procedure is being performed.

7. The ultrasound imaging system of claim 1, further comprising a controller configured to cause the ultrasound probe to cease transmitting ultrasound signals except for ultrasound signals required to generate the planar ultrasound image along the image plane relevant to the selected interventional medical procedure.

8. The ultrasound imaging system of claim 1, wherein the user interface is further configured to receive a confirmation that a step in the selected interventional medical procedure has been performed.

9. The ultrasound imaging system of claim 8, wherein the user interface is further configured to display a second planar ultrasound image based on the confirmation, the second planar ultrasound image being necessary to perform a subsequent step in the selected interventional medical procedure.

10. The ultrasound imaging system of claim 1, wherein the image reconstruction processor is configured to generate the planar ultrasound image along the image plane relevant to the selected interventional medical procedure in real time.

11. The ultrasound imaging system of claim 1, wherein the image reconstruction processor is configured to optimize a scan sequence for the image plane relevant to the selected interventional medical procedure in an interval.

12. The ultrasound imaging system of claim 1, wherein the anatomical recognition processor is further configured to generate an indication that the image data is insufficient to identify the anatomical landmark.

13. A method comprising:
receiving a user input indicating a selection of an interventional medical procedure;
obtaining image data by transmitting ultrasound signals at a target region and receiving echoes responsive to the ultrasound signals from the target region;
automatically identifying an anatomical landmark within the image data;
automatically generating a planar ultrasound image along an image plane relevant to the selected interventional medical procedure based on the anatomical landmark identified within the image data; and
displaying the planar ultrasound image during the interventional medical procedure.

14. The method of claim 13, wherein the interventional medical procedure comprises a cardiovascular valve clip procedure, an annuloplasty procedure, or a left atrial appendage occlusion procedure.

15. The method of claim 13, further comprising receiving an indication of procedure-specific implant locations.

16. The method of claim 13, further comprising ceasing obtaining image data except for the image data required to generate the planar ultrasound image along the image plane relevant to the selected interventional medical procedure.

17. The method of claim 13, further comprising receiving a confirmation that a step in the selected interventional medical procedure has been performed.

18. The method of claim 17, further comprising displaying a second planar ultrasound image based on the confirmation, the second planar ultrasound image being necessary to perform a subsequent step in the selected interventional medical procedure.

19. The method of claim 13, further comprising generating the planar ultrasound image along the image plane relevant to the selected interventional medical procedure in real time.

20. The ultrasound imaging system of claim 13, further comprising optimizing a scan sequence for the image plane relevant to the selected interventional medical procedure in an interval.
